(19) United States
(12) Patent Application Publication
(10) Pub. No.: US 2007/0004693 A1
(43) Pub. Date: Jan. 4, 2007

(54) Estrogen compositions for vaginal administration

(75) Inventors: David Woolfson, Belfast (IE); Claire Gilligan, Belfast (IE)

Correspondence Address: FITZPATRICK CELLA HARPER & SCINTO, 30 ROCKEFELLER PLAZA, NEW YORK, NY, 10112, US

(73) Assignee: WARNER CHILCOTT COMPANY INC., Fajardo, PR (US)

(21) Appl. No.: 11/454,473

(22) Filed: Jun. 16, 2006

Related U.S. Application Data
(60) Provisional application No. 60/691,440, filed on Jun. 16, 2005.

(51) Int. Cl. A61K 31/56; A61K 47/00

(52) U.S. Cl. 514/182; 514/781

(57) ABSTRACT

Pharmaceutical gel compositions containing estrogen for vaginal administration, as well as a method of making the same, are disclosed.

This application claims the benefit of U.S. Provisional Patent Application No. 60/691,440, filed Jun. 16, 2005, the entire disclosure of which is incorporated by reference herein.

### BACKGROUND OF THE INVENTION

1. Field of the Invention

This invention is directed to estrogen compositions for vaginal administration, wherein a portion of the estrogen is suspended, as well as to methods of making and administering the same.

2. Related Background Art

Conventional estrogen vaginal preparations comprise a two-phase emulsified system. Such conventional estrogen vaginal preparations have a significant hydrophobic oil or wax component, present either as the external or, more typically, the internal phase of the two-phase emulsified system, where water constitutes the other phase. Given the preferential solubility of estrogen in the hydrophobic phase, its availability for release into vaginal epithelial tissue upon vaginal application of such a preparation is, therefore, likely to be restricted.

Another problem encountered with conventional estrogen preparations for vaginal application is that they can be greasy and/or extremely difficult to remove completely from an applicator, particularly if the applicator is washed with water only.

The significant hydrophobic oil or wax component of conventional estrogen vaginal preparations contributes to both of these problems. Pharmaceutical gel compositions containing estrogen for vaginal administration may be advantageous for vaginal use. As used herein, the term “gel” is understood to be a semi-solid suspension of particles interpenetrated by a liquid, in which the structural coherent matrix contains a high portion of the liquid, usually an aqueous solvent such as water and/or water miscible solvents. Such gels comprise a single phase. As used herein, the term “semi-solid” is understood to refer to the rheological properties of the compositions themselves, such that the compositions will flow under an applied force but will remain in situ following application to the vaginal epithelial surface.

The inventors are not aware of any commercially available pharmaceutical gel compositions containing estrogen for vaginal administration. Known pharmaceutical gel compositions for topical administration conventionally contain an alcoholic component, ethanol, as an estrogen solubilizer. Ethanol is typically used as a penetration enhancer, especially in transdermal preparations, and exerts a drying action on skin and epithelial tissues due to solubilization of the hydrophobic components of the tissue. More specifically, known pharmaceutical gel compositions for topical, but not for vaginal, administration comprise Estrogel® (Solvay, US) and Sandrena® (Organon, Netherlands). Estrogel® is a hydro-ethanolic gel containing 0.06% estradiol; the excipients are ethanol, Carbomer 934 and triethanolamine, the balance being purified water. Sandrena® is another hydro-alcoholic gel containing 0.1% estradiol; its excipients are Carbomer 934, sodium hydroxide, propylene glycol, ethanol and water. Clearly, the base of these pharmaceutical gel compositions is a mixture of water and ethanol (and propylene glycol in the case of Sandrena®). The ethanol is intended to increase estrogen solubility in the gel and assist absorption into the stratum corneum. While the presence of ethanol may be useful in a topical or skin composition, its presence is counter-productive in mucosal utilities such as vaginal compositions since it is an irritant and may also have a drying effect, which is even more undesirable for those for whom such a product would be intended (post-menopausal women with vulval and vaginal atrophy).

Accordingly, pharmaceutical gel compositions containing estrogen for vaginal administration which do not suffer from the deficiencies of conventional vaginal preparations and topical gel compositions are highly desirable.

### SUMMARY OF THE INVENTION

The present invention is directed to a pharmaceutical gel composition containing estrogen for vaginal administration comprising (a) at least one estrogen in an amount of about 0.00028% to about 1% by weight of the composition; (b) at least one gelation polymer; and (c) at least one aqueous solvent, wherein a portion of the estrogen in the composition is in suspension at 15° C.

In certain embodiments of the present invention, the at least one estrogen is present in an amount ranging from about 0.0007% to about 0.05% by weight of the composition. In other preferred embodiments of this invention, at least 50%, more preferably at least 60%, and most preferably at least 90%, of the estrogen contained in the composition is in suspension at 15° C.

The at least one estrogen is preferably selected from 17β-estradiol, mestranol, conjugated estrogens USP, estrone, and ethinyl estradiol, and salts, esters and prodrugs thereof. The at least one gelation polymer is preferably selected from cellulose derivatives, gums, and neutralised homopolymers, copolymers and interpolymers having pendent carboxylic acid groups, or their esters, and/or having pendent anhydrides of dicarboxylic acid groups. The at least one aqueous solvent is preferably water, either alone or mixed with at least one water miscible solvent.

The present invention is further directed to a method of making a pharmaceutical gel composition for vaginal administration comprising the step of admixing at least one estrogen in an amount of about 0.00028% to about 1% by weight of the composition with at least one aqueous solvent and at least one gelation polymer to form the pharmaceutical gel composition, wherein a portion of the estrogen in the composition is in suspension at 15° C. In a preferred embodiment, the admixing comprises (a) mixing the estrogen in the aqueous solvent to form an estrogen suspension; and (b) combining the estrogen suspension with the gelation polymer to form the pharmaceutical gel composition.

The present invention is still further directed to a pharmaceutical gel composition made according to the present inventive method and to a method of administering the pharmaceutical gel composition of the present invention.

### BRIEF DESCRIPTION OF THE FIGURES

FIG. 1 illustrates the cumulative estradiol release of both emulsified oil in water preparations and pharmaceutical gel compositions containing suspended estrogen for vaginal administration of the present invention.

FIG. 2 illustrates the fractional release of estradiol of both emulsified oil in water preparations and pharmaceutical gel compositions containing suspended estrogen for vaginal administration of the present invention.

### DETAILED DESCRIPTION OF THE INVENTION

The first embodiment of the present invention is a pharmaceutical gel composition containing estrogen for vaginal administration comprising at least one estrogen; at least one gelation polymer; and at least one aqueous solvent, wherein a portion of the estrogen in the composition is in suspension at 15° C.

Any form of estrogen can be used for purposes of this invention. Such suitable forms of estrogen include, without limitation, 17β-estradiol, mestranol, conjugated estrogens USP, estrone, ethinyl estradiol, and combinations thereof, as well as salts, esters (such as 17β-estradiol-3-acetate) or prodrugs thereof. Other suitable estrogens include those described in each of U.S. patent application Ser. Nos. 11/009,617 and 11/009,618 [Attorney Docket Nos. 02911.004500 and 02911.004400, respectively], each filed on Dec. 10, 2004, and those described in each of U.S. Provisional Patent Application Nos. 60/698,865 and 60/698,866 [Attorney Docket Nos. 02911.006500 and 02911.006900, respectively], each filed on Jul. 12, 2005. The disclosures of each of these applications are incorporated in their entirety by reference herein. The estrogen is included in the pharmaceutical gel composition of the present invention in a total amount ranging from about 0.00028% to about 1%, more preferably about 0.0007% to about 0.05%, by weight of the composition.

Importantly, a large portion of the estrogen in the inventive pharmaceutical gel compositions is in suspension at 15° C. Preferably, at least 50% of the estrogen is suspended at 15° C. More preferably, at least 60% of the estrogen is suspended at 15° C. and most preferably, at least 90% of the estrogen is suspended at 15° C. The units % relate to % w/w, so that “at least 50%” means that at least half, by weight, of the added estrogen is in suspension in the composition at 15° C. For purposes of any of the first to fourth embodiments of this invention, it is to be understood that “suspended” can refer to either or both of suspension in the composition as a whole or suspension in the at least one aqueous solvent.

An exemplary method of determining the portion of estrogen in suspension relates to the solubility of the estrogen in the aqueous solvent at 15° C. The solubility is calculated by preparing saturated solutions of the estrogen in the aqueous solvent at 15° C. (in triplicate). These saturated solutions were prepared by adding the estrogen to the aqueous solvent at 15° C. until saturation was achieved (i.e., no more estrogen dissolved in the aqueous solvent). The saturated solutions were then placed in a shaker at 15° C. for 12 hours, after which time more estrogen was added if required. Finally, the saturated solutions were centrifuged at 15° C. and the supernatant analyzed by HPLC to determine the amount of dissolved estrogen in the aqueous solvent. The portion of the estrogen in suspension in the aqueous solvent is determined by subtracting the amount dissolved in the aqueous solvent from the initial amount added. The amount of dissolved estrogen in the composition is measured by first centrifuging the composition under centrifugation conditions sufficient to remove any suspended estrogen, and then extracting the estrogen from the supernatant using a suitable solvent (such as ethanol) or solvent mixture. The solvent extract is then analyzed by HPLC to determine the amount of dissolved estrogen in the composition. The portion of the estrogen in suspension in the composition is determined by subtracting the amount dissolved in the supernatant from the initial amount added.

Surprisingly, despite the known low solubility of estrogen in water, it has now been found that the compositions of the present invention can efficiently deliver estrogen in amounts known to be clinically efficient. Without being bound by theory, it is believed that, by maintaining a large portion of the estrogen component in a suspended state within the composition, the solid drug can act as a reservoir within the composition to replace dissolved drug within the composition that is released into vaginal fluid; in turn, drug saturation of vaginal fluid is maintained and a substantially linear release of drug is observed from the composition. This is in contrast to release mechanisms observed with conventional emulsified estrogen vaginal preparations or other topical estrogen gel formulations having at least one non-aqueous component that acts as an estrogen solubilizing agent, where partitioning between aqueous and non-aqueous phases of the composition thermodynamically favors drug retention in the non-aqueous phase, thus retarding release into vaginal fluid.

In view of the importance of suspended estrogen in compositions of the present invention, the pharmaceutical compositions are substantially free of estrogen solubilizing agents such as ethanol, propylene glycol, glycerol and the like. As used herein, the term “substantially free” is understood to be less than about 0.045% of said estrogen solubilizing agents, preferably less than about 0.005% of said estrogen solubilizing agents, more preferably less than about 0.001% of said estrogen solubilizing agents, by weight of the composition. In a particular embodiment, the term “substantially free” may be understood to be less than about 0.05% of ethanol, preferably less than about 0.005% of ethanol, still more preferably less than about 0.001% of ethanol, by weight of the composition. All % units are w/w.

Gelation polymers suitable for use in the present invention include pharmaceutical gelling agents known in the art. Those include, without limitation, cellulose derivatives such as hydroxyethylcellulose, hydroxypropylcellulose and hydroxypropylmethylcellulose, gums such as xanthan gum, neutralised homopolymers, copolymers and interpolymers having pendent carboxylic acid groups, or their esters, and/or having pendent anhydrides of dicarboxylic acid groups such as polyacrylic acid derivatives (e.g., those sold under the tradename Carbopol® (Noveon, US)) or polymethyl vinyl ether/maleic anhydride copolymers (e.g., those sold under the tradename Gantrez® (ISP, US)), and combinations thereof. The gelation polymer is included in the pharmaceutical composition of the present invention in an amount to give a viscosity of between about 50 Pa·s and about 1000 Pa·s at 20° C., and more preferably between about 80 Pa·s and about 300 Pa·s at 20° C.

The aqueous solvent of the present invention is preferably water or buffered aqueous solutions. However, other water miscible solvents which may be employed in combination with water include any which can maintain the ability of the estrogen to be suspended—in other words, a water miscible solvent which would increase the solubility of the at least one estrogen too much would be undesirable. The aqueous solvent is present in the semi-solid pharmaceutical composition of the present invention in an amount effective to gel the gelation polymer. A suitable amount of aqueous solvent can be readily determined by one of ordinary skill in the art.

The pharmaceutical compositions of the present invention may also contain other suitable active ingredients. Suitable active ingredients include, without limitation, other steroids such as a progestogen (for example, progestogen and its derivatives such as 17-hydroxy progestogen esters and 19-nor-17-hydroxy progestogen esters, norgestrel, norgestimate, demegestone, drospirenone, dydrogesterone, medrogestone, medroxy progesterone and esters thereof such as medroxy progesterone acetate, norethesterone, norethindrone, norethindrone acetate, levonorgestrel, desogestrel, 3-ketodesogestrel, gestodene and the like) or an androgen (such as testosterone, esters thereof, methyl-testosterone and prodrugs and combinations thereof), in an amount appropriate for clinical efficacy.

The pharmaceutical composition of the present invention may also contain any pharmaceutically acceptable excipient, as desired. When present, such pharmaceutically acceptable excipients are included in an amount which can be readily determined by one of ordinary skill in the art. Suitable excipients include, without limitation, poly(vinyl alcohol), waxes (such as white soft paraffin), suitable preservatives including, without limitation, para-hydroxy benzoate compounds, buffers (for example, those buffers comprising weak organic acids such as lactic acid or acetic acid) and combinations thereof.

The second embodiment of the present invention is directed to a method of making a pharmaceutical gel composition containing estrogen for vaginal administration comprising the step of admixing at least one estrogen in an amount of about 0.00028% to about 1% by weight of the composition with at least one aqueous solvent and at least one gelation polymer to form a pharmaceutical gel composition, wherein a portion of the estrogen in the composition is in suspension at 15° C. The ingredients can be admixed using any suitable means. Typically, any mixing step is accomplished in a suitable vessel with agitation. According to a preferred embodiment of the inventive method, estrogen is first suspended in the aqueous solvent and then the estrogen suspension is combined with at least one gelation polymer.

Optional additional steps include those which result in the addition of one or more of another active ingredient(s) and pharmaceutically acceptable excipient(s). The details regarding the estrogen, gelation polymer and aqueous solvent, i.e., type and amount, % suspended, etc., as well as the details regarding other possible ingredients, are as set forth above with regard to the first embodiment of this invention.

An additional embodiment of the present invention is directed to a pharmaceutical gel composition made according to the method of the second embodiment of the invention.

Still another embodiment of the present invention is directed to a method of vaginal administration of estrogen for a female comprising the step of administering the pharmaceutical gel composition of the present invention to the vaginal epithelial tissue of the female.

Further embodiments of the present invention are directed to pharmaceutical gel compositions having the same composition as those of the first embodiment of the invention, but which are intended for topical administration. “Topical administration” refers to administration onto any accessible body surface of any human or animal species, for example, the skin or mucosal epithelia, and especially refers to an external application to a skin surface.

Still further embodiments of the present invention are directed to pharmaceutical gel compositions having a similar composition to those of the first embodiment, but which utilize active ingredients other than estrogen and which are intended for either vaginal or topical administration. The active ingredient(s) may be any pharmacologically active ingredient, typically those that exhibit prophylactic, therapeutic or cosmetic activity.

Specific embodiments of the invention will now be demonstrated by reference to the following examples. It should be understood that these examples are disclosed solely by way of illustrating the invention and should not be taken in any way to limit the scope of the present invention.

### EXAMPLE 1

A pharmaceutical gel composition was made to contain the components set forth in Table 1 below.

TABLE 1

| Ingredient | % w/w |
|---|---|
| Hydroxyethylcellulose | 3.00 |
| (Natrosol, 250 HHX-Pharm) | |
| Water | q.s to 100 |
| 17β-estradiol | 0.01 |
| Methyl parabens | 0.08 |
| Propyl parabens | 0.02 |

The 17β-estradiol was suspended in water with constant stirring at 20° C. Then, the hydroxyethylcellulose was added. A single phase gelled system having a viscosity of 166 Pa · s at 20° C. was obtained. About 98% of the estrogen is in suspension at 15° C.

The gel viscosity above was determined using a TA Advanced Rheometer AR550 in stepped flow mode, with a time constant of 10 seconds. The sample was loaded between a set of 40 mm standard parallel plates, with a plate gap of 1000 microns. The sample was allowed to equilibrate for 2 minutes before the shear stress was applied. A fresh sample was applied for each replicate analysis. The shear stress was increased from 100-300 Pa, and the viscosity was determined by application of the Power Law Model to the resulting flow rheogram. All analyses were performed at a controlled temperature of 20° C. Three readings were performed, and an average viscosity calculated.

### EXAMPLE 2

A pharmaceutical gel composition was made to contain the components set forth in Table 2 below.

TABLE 2

| Ingredient | % w/w |
|---|---|
| Hydroxyethylcellulose | 3.000 |
| (Natrosol, 250 HHX-Pharm) | |
| Water | q.s. to 100 |
| 17β-estradiol | 0.0015 |
| Methyl parabens | 0.080 |
| Propyl parabens | 0.020 |

The 17β-estradiol was suspended in the water with constant stirring at 20° C. Then, the hydroxyethylcellulose was added. A single phase gelled system having a viscosity of 130 Pa · s at 20° C. was obtained (as determined using the method of Example 1). About 86% of the estrogen is in suspension at 15° C.

### COMPARATIVE EXAMPLE 1

Two different estrogen emulsified oil-in-water cream preparations (Creams A and B) were made as set forth in Table 3. Cream C is a control containing no estrogen.

TABLE 3

| | Emulsifying | | | Total |
|---|---|---|---|---|
| | ointment* | Estradiol | Purified water | estradiol |
| Composition | (% w/w) | (% w/w) | (% w/w) | (μg) in 5 g |
| A | 50.0 | 0.010 | 49.990 | 500 |
| B | 50.0 | 0.001 | 49.999 | 50 |
| C | 50.0 | 0 | 50.0 | 0 |
| (control) | | | | |

*30% w/w emulsifying wax BP, 50% w/w white soft paraffin, 20% w/w liquid paraffin BP, supplied by Pinewood Healthcare, Clonmel, Ireland.
Creams A, B and C are all two-phase systems and had roughly the same consistency as the single-phase gels of Examples 1 and 2 of the present invention.

### COMPARATIVE RELEASE TESTING

Each of the creams of Comparative Example 1 and each of the inventive gels of Examples 1 and 2 were stored in lacquered ointment tubes at 4° C. for a period of 24 hours. 5.00 g of each cream and each gel were each placed in corresponding modified perforated cellulose bags, which were then sealed with tape. The bags were then positioned in 100 ml of 1% w/w benzalkonium chloride solution at 37° C. and placed in an orbital shaking incubator (Gallenkamp IOC, 37° C., 60 RPM). Samples were taken from the benzalkonium chloride solution at 1 hour, 4 hours and 24 hours and analyzed by high-performance liquid chromatography (HPLC) using the following method:

column: Symmetry Shield™ RP18 5 μm 4.6×150 mm

mobile phase: 50:50 Acetonitrile:pH 2.5 phosphate buffer

flow rate: 0.8 ml/min

injection volume: 20 μl

wavelength: 225 nm

column temperature: 30° C.

The estradiol release results for each of the tested samples is shown in Tables 4 and 5 below.

TABLE 4
Estradiol release (μg).

| | | | Control |
|---|---|---|---|
| Time | Cream A | Example 1 | Cream C |
| Total amount | 500 | 500 | 0 |
| of estradiol | | | |
| (μg) in 5 g dose | | | |
| 1 | 22.76 | 9.42 | 0 |
| 4 | 54.50 | 92.93 | 0 |
| 24 | 157.23 | 340.54 | 0 |

TABLE 5
Estradiol release (μg).

| | | | Control |
|---|---|---|---|
| Time | Cream B | Example 2 | Cream C |
| Total amount | 50 | 50 | 0 |
| of estradiol | | | |
| (μg)in 5 g dose | | | |
| 1 | 0 | 0 | 0 |
| 4 | 5.08 | 6.71 | 0 |
| 24 | 14.57 | 29.85 | 0 |

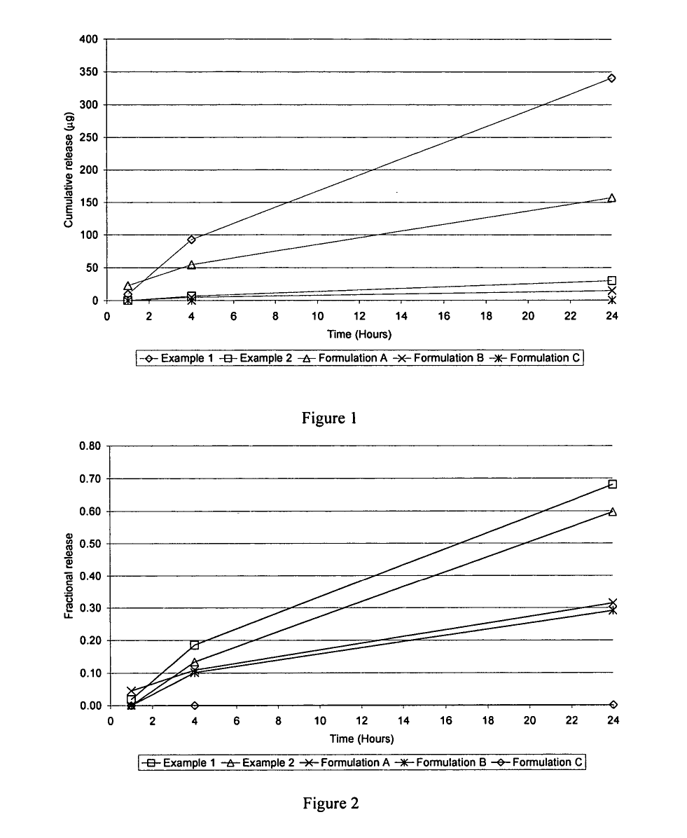

As can be seen from Table 4 above, Cream A released significantly less estradiol than the corresponding inventive pharmaceutical gel composition of Example 1 of the present invention. Similarly, Table 5 above shows that Cream B released significantly less estradiol than the corresponding inventive pharmaceutical gel composition of Example 2 of the present invention. This point is further illustrated by referring to FIG. 1 in which cumulative estradiol release is plotted for each of the tested samples. In addition, drug release is plotted as a fraction of total drug loading in FIG. 2. From this figure, it can easily be seen that the inventive pharmaceutical gel compositions were found to release over 100% more estradiol than their cream-based equivalents over a 24-hour period.

While the invention has been described above with reference to specific embodiments thereof, it is apparent that many changes, modifications, and variations can be made without departing from the inventive concept disclosed herein. Accordingly, it is intended to embrace all such changes, modifications, and variations that fall within the spirit and broad scope of the appended claims.

What is claimed is:

1. A pharmaceutical gel composition containing estrogen for vaginal administration comprising:
(a) at least one estrogen in an amount of about 0.00028% to about 1% by weight of the composition;
(b) at least one gelation polymer; and
(c) at least one aqueous solvent;
wherein a portion of the estrogen in the composition is in suspension at 15° C.

2. The pharmaceutical gel composition of claim 1, wherein the at least one estrogen is selected from the group consisting of 17β-estradiol, mestranol, conjugated estrogens USP, estrone, and ethinyl estradiol, and salts, esters, and prodrugs thereof.

3. The pharmaceutical gel composition of claim 1, wherein the amount of the at least one estrogen is from about 0.0007% to about 0.05% by weight of the composition.

4. The pharmaceutical gel composition of claim 1, wherein the at least one gelation polymer is selected from the group consisting of cellulose derivatives, gums, and neutralised homopolymers, copolymers and interpolymers having pendent carboxylic acid groups, or their esters, and/or having pendent anhydrides of dicarboxylic acid groups.

5. The pharmaceutical gel composition of claim 1, wherein the at least one gelation polymer is present in an amount sufficient to form a gel with a viscosity of between about 50 and about 1000 Pa·s at 20° C.

6. The pharmaceutical gel composition of claim 1, wherein the aqueous solvent is water.

7. The pharmaceutical gel composition of claim I, wherein at least 50% of the estrogen in the composition is in suspension at 15° C.

8. The pharmaceutical gel composition of claim 7, wherein at least 60% of the estrogen in the composition is in suspension at 15° C.

9. The pharmaceutical gel composition of claim 8, wherein at least 90% of the estrogen in the composition is in suspension at 15° C.

10. The pharmaceutical gel composition of claim 1, wherein the composition is substantially free of estrogen solubilizing agents.

11. The pharmaceutical gel composition of claim 1, further comprising at least one pharmaceutically acceptable excipient.

12. The pharmaceutical gel composition of claim 1, further comprising at least one active ingredient selected from the group consisting of progestogens and androgens.

13. The pharmaceutical gel composition of claim 12, wherein the progestogen is selected from the group consisting of progestogen, 17-hydroxy progestogen esters, 19-nor-17-hydroxy progestogen esters, norgestrel, norgestimate, desogestrel, demegestone, drospirenone, dydrogesterone, medrogestone, medroxy progesterone, medroxyprogesterone acetate, norethesterone, norethindrone, norethindrone acetate, levonorgestrel, 3-ketodesogestrel, gestodene and combinations thereof.

14. The pharmaceutical gel composition of claim 12, wherein the androgen is selected from the group consisting of testosterone, esters thereof, methyl-testosterone, prodrugs thereof and combinations thereof.

15. A method of making a pharmaceutical gel composition for vaginal application comprising the step of admixing at least one estrogen in an amount of about 0.00028% to about 1% by weight of the composition with at least one aqueous solvent and at least one gelation polymer to form the pharmaceutical gel composition, wherein a portion of the estrogen in the composition is in suspension at 15° C.

16. The method of claim 15, wherein the admixing step comprises (a) mixing the at least one estrogen with the at least one aqueous solvent to form an estrogen suspension and (b) combining the estrogen suspension with the at least one gelation polymer to form the pharmaceutical gel composition.

17. A pharmaceutical gel composition made according to the method of claim 15.

18. A method of vaginal administration of estrogen for a female comprising the step of administering the pharmaceutical gel composition of claim 1 to the vaginal epithelial tissue of the female.
